(19) United States
(12) Patent Application Publication
(10) Pub. No.: US 2004/0265833 A1
(43) Pub. Date: Dec. 30, 2004

(54) Methods and nucleic acids for the analysis of colorectal cell proliferative disorders

(76) Inventors: Cathy Lofton-Day, Brier, WA (US); Andrew Sledziewski, Shoreline, WA (US); Jeff Thomas, Bellevue, WA (US); Robert W. Day, Seattle, WA (US); Lori Tonnes-Priddy, Everett, WA (US); Karen Cardon, Renton, WA (US)

Correspondence Address: DAVIS WRIGHT TREMAINE, LLP, 2600 CENTURY SQUARE, 1501 FOURTH AVENUE, SEATTLE, WA, 98101-1688, US

(21) Appl. No.: 10/602,494

(22) Filed: Jun. 23, 2003

(51) Int. Cl. C12Q 1/68

(52) U.S. Cl. 435/6

(57) ABSTRACT

The present invention provides, inter alia, novel diagnostic and prognostic methods for detecting, or for detecting and differentiating between or among colorectal cell proliferative disorders. Preferably, said colorectal cell proliferative disorders are selected from the group consisting of colorectal carcinoma, colon adenomas, and colon polyps. The inventive methods are based on analysis of differential CpG dinucleotide methylation of genomic DNA between or among normal and disease states. Additional embodiments provide nucleic acids and oligomers (including oligonucleotides and peptide nucleic acid (PNA)-oligomers), nucleic acid arrays and kits useful for practicing said methods, and in otherwise detecting, or detecting and differentiating between or among colorectal cell proliferative disorders.

### FIELD OF THE INVENTION

The present invention relates to genomic DNA sequences that exhibit altered CpG methylation patterns in disease states relative to normal. Particular embodiments provide methods, nucleic acids, nucleic acid arrays and kits useful for detecting, or for detecting and differentiating between or among colorectal cell proliferative disorders.

### SEQUENCE LISTING

A Sequence Listing, pursuant to 37 C.F.R. § 1.52(e)(5), has been provided on compact disc (1 of 1) as a 1.436 MB file, entitled 47675-45.txt, and which is incorporated by reference herein in its entirety.

### BACKGROUND

The etiology of pathogenic states is known to involve modified methylation patterns of individual genes or of the genome. 5-methylcytosine, in the context of CpG dinucleotide sequences, is the most frequent covalently modified base in the DNA of eukaryotic cells, and plays a role in the regulation of transcription, genetic imprinting, and tumorigenesis. The identification and quantification of 5-methylcytosine sites in a specific specimen, or between or among a plurality of specimens, is thus of considerable interest, not only in research, but particularly for the molecular diagnoses of various diseases.

Correlation of Aberrant DNA Methylation with Cancer.

Aberrant DNA methylation within CpG ‘islands’ is characterized by hyper- or hypomethylation of CpG dinucleotide sequences leading to abrogation or overexpression of a broad spectrum of genes, and is among the earliest and most common alterations found in, and correlated with human malignancies. Additionally, abnormal methylation has been shown to occur in CpG-rich regulatory elements in intronic and coding parts of genes for certain tumors. In colon cancer, for example, aberrant DNA methylation constitutes one of the most prominent alterations and inactivates many tumor suppressor genes such as p14ARF, p16INK4a, THBS1, MINT2, and MINT31 and DNA mismatch repair genes such as hMLH1.

In contrast to the specific hypermethylation of tumor suppressor genes, an overall hypomethylation of DNA can be observed in tumor cells. This decrease in global methylation can be detected early, far before the development of frank tumor formation. A correlation between hypomethylation and increased gene expression has been determined for many oncogenes.

Colorectal Cancer.

Colorectal cancer is the fourth leading cause of cancer mortality in men and women, although ranking third in frequency in men and second in women. The 5-year survival rate is 61% over all stages with early detection being a prerequisite for curative therapy of the disease. Up to 95% of all colorectal cancers are adenocarcinomas of varying differentiation grades .

Sporadic colon cancer develops in a multistep process starting with the pathologic transformation of normal colonic epithelium to an adenoma which consecutively progresses to invasive cancer. The progression rate of benign colonic adenomas depends strongly on their histologic appearance: whereas tubular-type adenomas tend to progress to malignant tumors very rarely, villous adenomas, particularly if larger than 2 cm in diameter, have a significant malignant potential.

During progression from benign proliferative lesions to malignant neoplasms several genetic and epigenetic alterations occur. Somatic mutation of the APC gene seems to be one of the earliest events in 75 to 80% of colorectal adenomas and carcinomas. Activation of K-RAS is thought to be a critical step in the progression towards a malignant phenotype. Consecutively, mutations in other oncogenes as well as alterations leading to inactivation of tumor suppressor genes accumulate.

In the molecular evolution of colorectal cancer, DNA methylation errors have been suggested to play two distinct roles. In normal colonic mucosa cells, methylation errors accumulate as a function of age or as time-dependent events predisposing these cells to neoplastic transformation. For example, hypermethylation of several loci could be shown to be already present in adenomas, particularly in the tubulovillous and villous subtype. At later stages, increased DNA methylation of CpG islands plays an important role in a subset of tumors affected by the so called CpG island methylator phenotype (CIMP). Most CIMP+ tumors, which constitute about 15% of all sporadic colorectal cancers, are characterized by microsatellite instability (MIN) due to hypermethylation of the hMLH1 promoter and other DNA mismatch repair genes. By contrast, CIMP− colon cancers evolve along a more classic genetic instability pathway (CIN), with a high rate of p53 mutations and chromosomal changes.

However, the molecular subtypes do not only show varying frequencies regarding molecular alterations. According to the presence of either micro satellite instability or chromosomal aberrations, colon cancer can be subclassified into two classes, which also exhibit significant clinical differences. Almost all MIN tumors originate in the proximal colon (ascending and transversum), whereas 70% of CIN tumors are located in the distal colon and rectum. This has been attributed to the varying prevalence of different carcinogens in different sections of the colon. Methylating carcinogens, which constitute the prevailing carcinogen in the proximal colon have been suggested to play a role in the pathogenesis of MIN cancers, whereas CIN tumors are thought to be more frequently caused by adduct-forming carcinogens, which occur more frequently in distal parts of the colon and rectum. Moreover, MIN tumors have a better prognosis than do tumors with a CIN phenotype and respond better to adjuvant chemotherapy.

Incidence and mortality rates for this disease increase greatly with age, particularly after the age of 60. Stage of disease at diagnosis also affects overall survival rates. Patients having lesions confined to the colonic wall have a high probability of surviving 5 or more years while patients with metastatic disease have a very low probability of survival. It is thought that most colorectal cancers develop over a course of 5-10 years from a precursor lesion called an adenomatous polyp. The potential of these lesions to result in adenocarcinoma has been shown to increase with both polyp size and degree of dysplasia. Because of the slow progression of this disease, early detection through routine screening can result in significant improvement of survival rates. Several randomized trials over the last 20 years have shown that screening test can reduce mortality over 30%, even though the tests used were not highly sensitive. The current guidelines for colorectal screening according to the American Cancer Society utilizes one of five different options for screening in average risk individuals 50 years of age or older. These options include 1) fecal occult blood test (FOBT) annually, 2) flexible sigmoidoscopy every five years, 3) annual FPBT plus flexible sigmoidoscopy every five years, 4) double contrast barium enema (DCBE) every five years or 5) colonoscopy every ten years. Even though these testing procedures are well accepted by the medical community, the implementation of widespread screening for colorectal cancer has not been realized. Patient compliance is a major factor for limited use due to the discomfort or inconvenience associated with the procedures. FOBT testing, although a non-invasive procedure, requires dietary and other restrictions 3-5 days prior to testing. Sensitivity levels for this test are also very low for colorectal adenocarcinoma with wide variability depending on the trial. Sensitivity measurements for detection of adenomas is even less since most adenomas do not bleed. In contrast, sensitivity for more invasive procedures such as sigmoidoscopy and colonoscopy are quite high because of direct visualization of the lumen of the colon. No randomized trials have evaluated the efficacy of these techniques, however, using data from case-control studies and data from the National Polyp Study (U.S.) it has been shown that removal of adenomatous polyps results in a 76-90% reduction in CRC incidence. Sigmoidoscopy has the limitation of only visualizing the left side of the colon leaving lesions in the right colon undetected. Both scoping procedures are expensive, require cathartic preparation and have increased risk of morbidity and mortality. Improved tests with increased sensitivity, specificity, ease of use and decreased costs are clearly needed before general widespread screening for colorectal cancer becomes routine.

Molecular disease markers offer several advantages over other types of markers, one advantage being that even samples of very small sizes and/or samples whose tissue architecture has not been maintained can be analyzed quite efficiently. Within the last decade a number of genes have been shown to be differentially expressed between normal and colon carcinomas. However, no single or combination of marker has been shown to be sufficient for the diagnosis of colon carcinomas. High-dimensional mRNA based approaches have recently been shown to be able to provide a better means to distinguish between different tumor types and benign and malignant lesions. However its application as a routine diagnostic tool in a clinical environment is impeded by the extreme instability of mRNA, the rapidly occurring expression changes following certain triggers (e.g., sample collection), and, most importantly, the large amount of mRNA needed for analysis (Lipshutz, R. J. et al., Nature Genetics 21:20-24, 1999; Bowtell, D. D. L. Nature genetics suppl. 21:25-32, 1999), which often cannot be obtained from a routine biopsy.

There is a need in the art for a sensitive diagnostic or prognostic assay for colon cell proliferative disorders that is based, at least in part, on detection of differential methylation of CpG dinucleotide sequences, and that has a diagnostic or prognostic accuracy of greater than about 80%, preferably greater than about 85% or about 90%, more preferably greater than about 95%, and most preferably greater than about 98%.

### SUMMARY OF THE INVENTION

The present invention provides novel methods and nucleic acids useful for detecting, or detecting and distinguishing between or among colorectal cell proliferative disorders, most preferrably colorectal carcinoma, colon adenomas and colon polyps. The invention provides a method for the analysis of biological samples for features associated with the development of colon cell proliferative disorders, the method characterised in that at least one nucleic acid, or a fragment thereof, from the group consisting of SEQ ID NO:1 to SEQ ID NO:355 is/are contacted with a reagent or series of reagents capable of distinguishing between methylated and non methylated CpG dinucleotides within the genomic sequence, or sequences of interest.

The present invention provides a method for ascertaining genetic and/or epigenetic parameters of genomic DNA. The method has utility for the improved diagnosis, treatment and monitoring of colon cell proliferative disorders, more specifically by enabling the improved identification of, and differentiation between or among subclasses of said disorders and the genetic predisposition to said disorders. The invention presents improvements over the art in that, inter alia, it enables an accurate and highly specific classification of colon cell proliferative disorders, thereby allowing for improved and informed treatment of patients.

Preferably, the source of the test sample is selected from the group consisting of cells or cell lines, histological slides, biopsies, paraffin-embedded tissue, bodily fluids, ejaculate, urine, blood, and combinations thereof. Preferably, the source is biopsies, bodily fluids, ejaculate, urine, or blood.

Specifically, the present invention provides a method for detecting colon cell proliferative disorders, comprising: obtaining a biological sample comprising genomic nucleic acid(s); contacting the nucleic acid(s), or a fragment thereof, with one reagent or a plurality of reagents sufficient for distinguishing between methylated and non methylated CpG dinucleotide sequences within a target sequence of the subject nucleic acid, wherein the target sequence comprises, or hybridizes under stringent conditions to, a sequence comprising at least 18 contiguous nucleotides of a sequence selected from the group consisting of SEQ ID NO:1 to 355; and determining, based at least in part on said distinguishing, the methylation state of at least one target CpG dinucleotide sequence, or an average, or a value reflecting an average methylation state of a plurality of target CpG dinucleotide sequences. Preferably, the contiguous nucleotides comprise at least one CpG dinucleotide sequence. Preferably, distinguishing between methylated and non methylated CpG dinucleotide sequences within the target sequence comprises methylation state-dependent conversion or non-conversion of at least one such CpG dinucleotide sequence to the corresponding converted or non-converted dinucleotide sequence within a sequence selected from the group consisting of SEQ ID NO:72 to SEQ ID NO:355, and contiguous regions thereof corresponding to the target sequence.

Additional embodiments provide a method for the detection of colon cell proliferative disorders, comprising: obtaining a biological sample having subject genomic DNA; extracting, or otherwise isolating the genomic DNA; treating the extracted or otherwise isolated genomic DNA, or a fragment thereof, with one or more reagents to convert 5-position unmethylated cytosine bases to uracil or to another base that is detectably dissimilar to cytosine in terms of hybridization properties; contacting the treated genomic DNA, or the treated fragment thereof, with an amplification enzyme and at least two primers comprising, in each case a contiguous sequence at least 9 nucleotides in length that is complementary to, or hybridizes under moderately stringent or stringent conditions to a sequence selected from the group consisting SEQ ID NO:72 to SEQ ID NO: 355, and complements thereof, wherein the treated DNA or the fragment thereof is either amplified to produce an amplificate, or is not amplified; and determining, based on a presence or absence of, or on a property of said amplificate, the methylation state of at least one CpG dinucleotide sequence selected from the group consisting of SEQ ID NO: 1 to SEQ ID NO: 71, or an average, or a value reflecting an average methylation state of a plurality of CpG dinucleotide sequences thereof Preferably, at least one such hybridizing nucleic acid molecule or peptide nucleic acid molecule is bound to a solid phase. Further embodiments provide a method for the analysis of colon cell proliferative disorders, comprising: obtaining a biological sample having subject genomic DNA; extracting, or otherwise isolating the genomic DNA; contacting the extracted or otherwise isolated genomic DNA, or a fragment thereof, comprising one or more sequences selected from the group consisting of SEQ ID NO:1 to SEQ ID NO:71 or a sequence that hybridizes under stringent conditions thereto, with one or more methylation-sensitive restriction enzymes, wherein the genomic DNA is either digested thereby to produce digestion fragments, or is not digested thereby; and determining, based on a presence or absence of, or on property of at least one such fragment, the methylation state of at least one CpG dinucleotide sequence of one or more sequences selected from the group consisting of SEQ ID NO:1 to SEQ ID NO:71, or an average, or a value reflecting an average methylation state of a plurality of CpG dinucleotide sequences thereof. Preferably, the digested or undigested genomic DNA is amplified prior to said determining.

Additional embodiments provide novel genomic and chemically modified nucleic acid sequences, as well as oligonucleotides and/or PNA-oligomers for analysis of cytosine methylation patterns within sequences from the group consisting of SEQ ID NO:1 to SEQ ID NO:71.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 represents the sequencing data for a fragment of SEQ ID NO:46 according to EXAMPLE 2 herein below. Each row of the matrix represents a single CpG dinucleotide site within the fragment and each column is an individual DNA sample (sample designations are listed on the X-axis). The vertical calibration bar on the left correlates the intensity of shading or color with the percent of methylation; with the degree of methylation represented by the darkness of each position within the column from black (or blue) representing 100% methylation to light grey(or yellow) representing 0% methylation. Colon cancer samples are to the left of the central vertical black line and healthy colon samples are to the right of the vertical black line.

FIG. 2 represents the sequencing data for a fragment of SEQ ID NO: 14 according to EXAMPLE 2 herein below. Each row of the matrix represents a single CpG site within the fragment and each column is an individual DNA sample (sample designations are listed on the X-axis). The vertical calibration bar on the left correlates the intensity of shading or color with the percent of methylation; with the degree of methylation represented by the darkness of each position within the column from black (or blue) representing 100% methylation to light grey(or yellow) representing 0% methylation. Colon cancer samples are to the left of the central vertical black line and healthy colon samples are to the right of the central vertical black line.

FIG. 3 represents the sequencing data for a fragment of SEQ ID NO:69 according to EXAMPLE 2 herein below. Each row of the matrix represents a single CpG site within the fragment and each column is an individual DNA sample (sample designations are listed on the X-axis). The vertical calibration bar on the left correlates the intensity of shading or color with the percent of methylation; with the degree of methylation represented by the darkness of each position within the column from black (or blue) representing 100% methylation to light grey(or yellow) representing 0% methylation. Colon cancer samples are to the left of the left vertical black line, healthy colon samples are grouped between the left and right black lines, and peripheral blood lymphocytes (PBL) are grouped to the right of the right black vertical line.

### DETAILED DESCRIPTION OF THE INVENTION

Definitions:

The term “Observed/Expected Ratio” (“O/E Ratio”) refers to the frequency of CpG dinucleotides within a particular DNA sequence, and corresponds to the [number of CpG sites/(number of C bases×number of G bases)]×band length for each fragment.

The term “CpG island” refers to a contiguous region of genomic DNA that satisfies the criteria of (1) having a frequency of CpG dinucleotides corresponding to an “Observed/Expected Ratio” >0.6, and (2) having a “GC Content” >0.5. CpG islands are typically, but not always, between about 0.2 to about 1 kb, or to about 2 kb in length.

The term “methylation state” or “methylation status” refers to the presence or absence of 5-methylcytosine (“5-mCyt”) at one or a plurality of CpG dinucleotides within a DNA sequence. Methylation states at one or more particular palindromic CpG methylation sites (each having two CpG CpG dinucleotide sequences) within a DNA sequence include “unmethylated,” “fully-methylated” and “hemi-methylated.”

The term “hemi-methylation” or “hemimethylation” refers to the methylation state of a palindromic CpG methylation site, where only a single cytosine in one of the two CpG dinucleotide sequences of the palindromic CpG methylation site is methylated (e.g., 5′-CC^{M}GG-3′ (top strand): 3′-GGCC-5′ (bottom strand)).

The term “hypermethylation” refers to the average methylation state corresponding to an increased presence of 5-mCyt at one or a plurality of CpG dinucleotides within a DNA sequence of a test DNA sample, relative to the amount of 5-mCyt found at corresponding CpG dinucleotides within a normal control DNA sample.

The term “hypomethylation” refers to the average methylation state corresponding to a decreased presence of 5-mCyt at one or a plurality of CpG dinucleotides within a DNA sequence of a test DNA sample, relative to the amount of 5-mCyt found at corresponding CpG dinucleotides within a normal control DNA sample.

The term “microarray” refers broadly to both “DNA microarrays,” and ‘DNA chip(s),’ as recognized in the art, encompasses all art-recognized solid supports, and encompasses all methods for affixing nucleic acid molecules thereto or synthesis of nucleic acids thereon.

“Genetic parameters” are mutations and polymorphisms of genes and sequences further required for their regulation. To be designated as mutations are, in particular, insertions, deletions, point mutations, inversions and polymorphisms and, particularly preferred, SNPs (single nucleotide polymorphisms).

“Epigenetic parameters” are, in particular, cytosine methylations. Further epigenetic parameters include, for example, the acetylation of histones which, however, cannot be directly analyzed using the described method but which, in turn, correlate with the DNA methylation.

The term “bisulfite reagent” refers to a reagent comprising bisulfite, disulfite, hydrogen sulfite or combinations thereof, useful as disclosed herein to distinguish between methylated and unmethylated CpG dinucleotide sequences.

The term “Methylation assay” refers to any assay for determining the methylation state of one or more CpG dinucleotide sequences within a sequence of DNA.

The term “MS.AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, and described by Gonzalgo et al., Cancer Research 57:594-599, 1997.

The term “MethyLight™” refers to the art-recognized fluorescence-based real-time PCR technique described by Eads et al., Cancer Res. 59:2302-2306, 1999.

The term “HeavyMethyl™” assay, in the embodiment thereof implemented herein, refers to a HeavyMethyl™ MethylLight™ assay, which is a variation of the MethylLight™ assay, wherein the MethylLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones, Nucleic Acids Res. 25:2529-2531, 1997.

The term “MSP” (Methylation-specific PCR) refers to the art-recognized methylation assay described by Herman et al. Proc. Natl. Acad. Sci. USA 93:9821-9826, 1996, and by U.S. Pat. No. 5,786,146.

The term “COBRA” (Combined Bisulfite Restriction Analysis) refers to the art-recognized methylation assay described by Xiong & Laird, Nucleic Acids Res. 25:2532-2534, 1997.

The term “MCA” (Methylated CpG Island Amplification) refers to the methylation assay described by Toyota et al., Cancer Res. 59:2307-12, 1999, and in WO 00/26401A1.

The term “hybridization” is to be understood as a bond of an oligonucleotide to a complementary sequence along the lines of the Watson-Crick base pairings in the sample DNA, forming a duplex structure.

“Stringent hybridization conditions,” as defined herein, involve hybridizing at 68° C. in 5×SSC/5× Denhardt's solution/1.0% SDS, and washing in 0.2×SSC/0.1% SDS at room temperature, or involve the art-recognized equivalent thereof (e.g., conditions in which a hybridization is carried out at 60° C. in 2.5×SSC buffer, followed by several washing steps at 37° C. in a low buffer concentration, and remains stable). Moderately stringent conditions, as defined herein, involve including washing in 3×SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

The terms “array SEQ ID NO,” “composite array SEQ ID NO,” or “composite array sequence” refer to a sequence, hypothetical or otherwise, consisting of a head-to-tail (5′ to 3′) linear composite of all individual contiguous sequences of a subject array (e.g., a head-to-tail composite of SEQ ID NOS:1-71, in that order).

The terms “array SEQ ID NO node,” “composite array SEQ ID NO node,” or “composite array sequence node” refer to a junction between any two individual contiguous sequences of the “array SEQ ID NO,” the “composite array SEQ ID NO,” or the “composite array sequence.”

In reference to composite array sequences, the phrase “contiguous nucleotides” refers to a contiguous sequence region of any individual contiguous sequence of the composite array, but does not include a region of the composite array sequence that includes a “node,” as defined herein above.

Overview:

The present invention provides for molecular genetic markers that have novel utility for the analysis of methylation patterns associated with the development of colon cell proliferative disorders. Said markers may be used for detecting, or for detecting and distinguishing between or among colon cell proliferative disorders.

Bisulfite Modification of DNA is an Art-Recognized Tool used to Assess CpG Methylation Status.

5-methylcytosine is the most frequent covalent base modification in the DNA of eukaryotic cells. It plays a role, for example, in the regulation of the transcription, in genetic imprinting, and in tumorigenesis. Therefore, the identification of 5-methylcytosine as a component of genetic information is of considerable interest. However, 5-methylcytosine positions cannot be identified by sequencing, because 5-methylcytosine has the same base pairing behavior as cytosine. Moreover, the epigenetic information carried by 5-methylcytosine is completely lost during, e.g., PCR amplification.

The most frequently used method for analyzing DNA for the presence of 5-methylcytosine is based upon the specific reaction of bisulfite with cytosine whereby, upon subsequent alkaline hydrolysis, cytosine is converted to uracil which corresponds to thymine in its base pairing behavior. Significantly, however, 5-methylcytosine remains unmodified under these conditions. Consequently, the original DNA is converted in such a manner that methylcytosine, which originally could not be distinguished from cytosine by its hybridization behavior, can now be detected as the only remaining cytosine using standard, art-recognized molecular biological techniques, for example, by amplification and hybridization, or by sequencing. All of these techniques are based on differential base pairing properties, which can now be fully exploited.

The prior art, in terms of sensitivity, is defined by a method comprising enclosing the DNA to be analyzed in an agarose matrix, thereby preventing the diffusion and renaturation of the DNA (bisulfite only reacts with single-stranded DNA), and replacing all precipitation and purification steps with fast dialysis (Olek A, et al., A modified and improved method for bisulfite based cytosine methylation analysis, Nucleic Acids Res. 24:5064-6, 1996). It is thus possible to analyze individual cells for methylation status, illustrating the utility and sensitivity of the method. An overview of art-recognized methods for detecting 5-methylcytosine is provided by Rein, T., et al., Nucleic Acids Res., 26:2255, 1998.

The bisulfite technique, barring few exceptions (e.g., Zeschnigk M, et al., Eur J Hum Genet. 5:94-98, 1997), is currently only used in research. In all instances, short, specific fragments of a known gene are amplified subsequent to a bisulfite treatment, and either completely sequenced (Olek & Walter, Nat Genet. 1997 17:275-6, 1997), subjected to one or more primer extension reactions (Gonzalgo & Jones, Nucleic Acids Res., 25:2529-31, 1997; WO 95/00669; U.S. Pat. No. 6,251,594) to analyze individual cytosine positions, or treated by enzymatic digestion (Xiong & Laird, Nucleic Acids Res., 25:2532-4, 1997). Detection by hybridization has also been described in the art (Olek et al., WO 99/28498). Additionally, use of the bisulfite technique for methylation detection with respect to individual genes has been described (Grigg & Clark, Bioessays, 16:431-6, 1994; Zeschnigk M, et al., Hum Mol Genet., 6:387-95, 1997; Feil R, et al., Nucleic Acids Res., 22:695-, 1994; Martin V, et al., Gene, 157:261-4, 1995; WO 9746705 and WO 9515373).

The present invention provides for the use of the bisulfite technique for determination of the methylation status of CpG dinuclotide sequences within genomic sequences from the group consisting of SEQ ID NO:1 to SEQ ID NO:71. According to the present invention, determination of the methylation status of CpG dinuclotide sequences within sequences from the group consisting of SEQ ID NO:1 to SEQ ID NO:71 has diagnostic and prognostic utility.

Methylation Assay Procedures.

Various methylation assay procedures are known in the art, and can be used in conjunction with the present invention. These assays allow for determination of the methylation state of one or a plurality of CpG dinucleotides (e.g., CpG islands) within a DNA sequence. Such assays involve, among other techniques, DNA sequencing of bisulfite-treated DNA, PCR (for sequence-specific amplification), Southern blot analysis, and use of methylation-sensitive restriction enzymes.

For example, genomic sequencing has been simplified for analysis of DNA methylation patterns and 5-methylcytosine distribution by using bisulfite treatment (Frommer et al., Proc. Natl. Acad. Sci. USA 89:1827-1831, 1992). Additionally, restriction enzyme digestion of PCR products amplified from bisulfite-converted DNA is used, e.g., the method described by Sadri & Hornsby (Nucl. Acids Res. 24:5058-5059, 1996), or COBRA (Combined Bisulfite Restriction Analysis) (Xiong & Laird, Nucleic Acids Res. 25:2532-2534, 1997).

COBRA.

COBRA analysis is a quantitative methylation assay useful for determining DNA methylation levels at specific gene loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the interested CpG islands, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples. Typical reagents (e.g., as might be found in a typical COBRA-based kit) for COBRA analysis may include, but are not limited to: PCR primers for specific gene (or methylation-altered DNA sequence or CpG island); restriction enzyme and appropriate buffer; gene-hybridization oligo; control hybridization oligo; kinase labeling kit for oligo probe; and radioactive nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

Preferably, assays such as “MethyLight™” (a fluorescence-based real-time PCR technique) (Eads et al., Cancer Res. 59:2302-2306, 1999), Ms-SNuPE (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with other of these methods.

MethyLight™.

The MethyLight™ assay is a high-throughput quantitative methylation assay that utilizes fluorescence-based real-time PCR (TaqMan®) technology that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed either in an “unbiased” (with primers that do not overlap known CpG methylation sites) PCR reaction, or in a “biased” (with PCR primers that overlap known CpG dinucleotides) reaction. Sequence discrimination can occur either at the level of the amplification process or at the level of the fluorescence detection process, or both.

The MethyLight™ assay may be used as a quantitative test for methylation patterns in the genomic DNA sample, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe overlie any CpG dinucleotides. Alternatively, a qualitative test for genomic methylation is achieved by probing of the biased PCR pool with either control oligonucleotides that do not “cover” known methylation sites (a fluorescence-based version of the “MSP” technique), or with oligonucleotides covering potential methylation sites.

The MethyLight™ process can by used with a “TaqMan®” probe in the amplification process. For example, double-stranded genomic DNA is treated with sodium bisulfite and subjected to one of two sets of PCR reactions using TaqMan® probes; e.g., with either biased primers and TaqMan® probe, or unbiased primers and TaqMan® probe. The TaqMan® probe is dual-labeled with fluorescent “reporter” and “quencher” molecules, and is designed to be specific for a relatively high GC content region so that it melts out at about 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific gene (or methylation-altered DNA sequence or CpG island); TaqMan® probes; optimized PCR buffers and deoxynucleotides; and Taq polymerase.

Ms-SNuPE.

The Ms-SNuPE technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest. Small amounts of DNA can be analyzed (e.g., microdissected pathology sections), and it avoids utilization of restriction enzymes for determining the methylation status at CpG sites.

Typical reagents (e.g., as might be found in a typical Ms-SNuPE-based kit) for Ms-SNuPE analysis may include, but are not limited to: PCR primers for specific gene (or methylation-altered DNA sequence or CpG island); optimized PCR buffers and deoxynucleotides; gel extraction kit; positive control primers; Ms-SNuPE primers for specific gene; reaction buffer (for the Ms-SNuPE reaction); and radioactive nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

MSP.

MSP (methylation-specific PCR) allows for assessing the methylation status of virtually any group of CpG sites within a CpG island, independent of the use of methylation-sensitive restriction enzymes (Herman et al. Proc. Natl. Acad Sci. USA 93:9821-9826, 1996; U.S. Pat. No. 5,786,146). Briefly, DNA is modified by sodium bisulfite converting all unmethylated, but not methylated cytosines to uracil, and subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific gene (or methylation-altered DNA sequence or CpG island), optimized PCR buffers and deoxynucleotides, and specific probes.

MCA.

The MCA technique is a method that can be used to screen for altered methylation patterns in genomic DNA, and to isolate specific sequences associated with these changes (Toyota et al., Cancer Res. 59:2307-12, 1999); Briefly, restriction enzymes with different sensitivities to cytosine methylation in their recognition sites are used to digest genomic DNAs from primary tumors, cell lines, and normal tissues prior to arbitrarily primed PCR amplification. Fragments that show differential methylation are cloned and sequenced after resolving the PCR products on high-resolution polyacrylamide gels. The cloned fragments are then used as probes for Southern analysis to confirm differential methylation of these regions. Typical reagents (e.g., as might be found in a typical MCA-based kit) for MCA analysis may include, but are not limited to: PCR primers for arbitrary priming Genomic DNA; PCR buffers and nucleotides, restriction enzymes and appropriate buffers; gene-hybridization oligos or probes; control hybridization oligos or probes.

Genomic Sequences According To SEQ. ID NO:1 to SEQ ID NO:71, and Treated Variants Thereof According to SEQ ID NO:72 to SEQ ID NO:355, Were Determined to have Utility for the Detection, Classification and/or Treatment of Colon Cell Proliferative Disorders

The present invention is based upon the analysis of methylation levels within one or more genomic sequences taken from the group consisting SEQ ID NO:1 to SEQ ID NO:71.

Particular embodiments of the present invention provide a novel application of the analysis of methylation levels and/or patterns within said sequences that enables a precise detection, characterisation and/or treatment of colon cell proliferative disorders. Early detection of colon cell proliferative disorders is directly linked with disease prognosis, and the disclosed method thereby enables the physician and patient to make better and more informed treatment decisions.

### FURTHER IMPROVEMENTS

The present invention provides novel uses for genomic sequences selected from the group consisting of SEQ ID NO:1 to SEQ ID NO:71. Additional embodiments provide modified variants of SEQ ID NO:1 to SEQ ID NO:71, as well as oligonucleotides and/or PNA-oligomers for analysis of cytosine methylation patterns within SEQ ID NO:1 to SEQ ID NO:71 .

An objective of the invention comprises analysis of the methylation state of one or more CpG dinucleotides within at least one of the genomic sequences selected from the group consisting of SEQ ID NO:1 to SEQ ID NO:71 and sequences complementary thereto.

In a preferred embodiment of the method, the objective comprises analysis of a modified nucleic acid comprising a sequence of at least 18 contiguous nucleotide bases in length of a sequence selected from the group consisting of SEQ ID NO:72 to SEQ ID NO:355, wherein said sequence comprises at least one CpG, TpA or CpA dinucleotide and sequences complementary thereto. The sequences of SEQ ID NO:72 to SEQ ID NO:355 provide modified versions of the nucleic acid according to SEQ ID NO:1 to SEQ ID NO:71, wherein the modification of each genomic sequence results in the synthesis of a nucleic acid having a sequence that is unique and distinct from said genomic sequence as follows:

For each sense strand genomic DNA, e.g., sense strand of SEQ ID NO:1, four converted versions are disclosed. A first version wherein “C”→“T,” but “CpG” remains “CpG” (i.e., corresponds to a case where, for the genomic sequence, all “C” residues of CpG dinucleotide sequences are methylated and are thus not converted); a second version discloses the complement of the disclosed genomic DNA sequence (i.e., antisense strand), wherein “C”→“T,” but “CpG” remains “CpG” (ie., corresponds to a case where, for all “C” residues of CpG dinucleotide sequences are methylated and are thus not converted). The ‘upmethylated’ converted sequences of SEQ ID NO:1 to SEQ ID NO:71 correspond to SEQ ID NO:72 to SEQ ID NO:213. A third chemically converted version of each genomic sequences is provided, wherein “C”→“T” for all “C” residues, including those of “CpG” dinucleotide sequences (i.e., corresponds to a case where, for the genomic sequences, all “C” residues of CpG dinucleotide sequences are unmethylated); and a final chemically converted version of each sequence, discloses the complement of the disclosed genomic DNA sequence (i.e., antisense strand), wherein “C”→“T” for all “C” residues, including those of “CpG” dinucleotide sequences (ie., corresponds to acase where, for the complement (antisense strand) of each genomic sequence, all “C” residues of CpG dinucleotide sequences are unmethylated). The ‘downmethylated’ converted sequences of SEQ ID NO:1 to SEQ ID NO:71 correspond to SEQ ID NO:214 to SEQ ID NO:355.

Significantly, heretofore, the nucleic acid sequences and molecules according to SEQ ID NO:1 to SEQ ID NO:355 were not implicated in or connected with the detection, classification or treatment of colon cell proliferative disorders.

In an alternative preferred embodiment, such analysis comprises the use of an oligonucleotide or oligomer for detecting the cytosine methylation state within genomic or pretreated (chemically modified) DNA, according to SEQ ID NO:1 to SEQ ID NO:355. Said oligonucleotide or oligomer comprising a nucleic acid sequence having a length of at least nine (9) nucleotides which hybridizes, under moderately stringent or stringent conditions (as defined herein above), to a pretreated nucleic acid sequence according to SEQ ID NO:72 to SEQ ID NO:355 and/or sequences complementary thereto, or to a genomic sequence according to SEQ ID NO:1 to SEQ ID NO:71 and/or sequences complementary thereto.

Thus, the present invention includes nucleic acid molecules, including oligomers (e.g., oligonucleotides and peptide nucleic acid (PNA) molecules (PNA-oligomers)) that hybridize under moderately stringent and/or stringent hybridization conditions to all or a portion of the sequences SEQ ID NO:1 to SEQ ID NO:355, or to the complements thereof. The hybridizing portion of the hybridizing nucleic acids is typically at least 9, 15, 20, 25, 30 or 35 nucleotides in length. However, longer molecules have inventive utility, and are thus within the scope of the present invention.

Preferably, the hybridizing portion of the inventive hybridizing nucleic acids is at least 95%, or at least 98%, or 100% identical to the sequence, or to a portion thereof of SEQ ID NO:1 to SEQ ID NO:355, or to the complements thereof.

Hybridizing nucleic acids of the type described herein can be used, for example, as a primer (e.g., a PCR primer), or a diagnostic and/or prognostic probe or primer. Preferably, hybridization of the oligonucleotide probe to a nucleic acid sample is performed under stringent conditions and the probe is 100% identical to the target sequence. Nucleic acid duplex or hybrid stability is expressed as the melting temperature or Tm, which is the temperature at which a probe dissociates from a target DNA. This melting temperature is used to define the required stringency conditions.

For target sequences that are related and substantially identical to the corresponding sequence of SEQ ID NO:1 to SEQ ID NO:71 (such as allelic variants and SNPs), rather than identical, it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch.

Examples of inventive oligonucleotides of length X (in nucleotides), as indicated by polynucleotide positions with reference to, e.g., SEQ ID NO: 1, include those corresponding to sets (sense and antisense sets) of consecutively overlapping oligonucleotides of length X, where the oligonucleotides within each consecutively overlapping set (corresponding to a given X value) are defined as the finite set of Z oligonucleotides from nucleotide positions:

n to(n+(X−1));

where n=1, 2, 3, . . . (Y−(X−1));

where Y equals the length (nucleotides or base pairs) of SEQ ID NO:1 (2,280);

where X equals the common length (in nucleotides) of each oligonucleotide in the set (e.g., X=20 for a set of consecutively overlapping 20-mers); and

where the number (Z) of consecutively overlapping oligomers of length X for a given SEQ ID NO of length Y is equal to Y−(X−1). For example Z=2,280-19=2,261 for either sense or antisense sets of SEQ ID NO:1, where X=20.

Preferably, the set is limited to those oligomers that comprise at least one CpQ, TpG or CpA dinucleotide.

Examples of inventive 20-mer oligonucleotides include the following set of 2,261 oligomers (and the antisense set complementary thereto), indicated by polynucleotide positions with reference to SEQ ID NO:1:

1-20, 2-21, 3-22, 4-23, 5-24, . . . 2259-2278, 2260-2279 and 2261-2280.

Preferably, the set is limited to those oligomers that comprise at least one CpG, TpG or CpA dinucleotide.

Likewise, examples of inventive 25-mer oligonucleotides include the following set of 2,256 oligomers (and the antisense set complementary thereto), indicated by polynucleotide positions with reference to SEQ ID NO:1:

1-25, 2-26, 3-27, 4-28, 5-29, . . . 2254-2278, 2255-2279 and 2256-2280.

Preferably, the set is limited to those oligomers that comprise at least one CpG, TpG or CpA dinucleotide.

The present invention encompasses, for each of SEQ ID NO:1 to SEQ ID NO:355 (sense and antisense), multiple consecutively overlapping sets of oligonucleotides or modified oligonucleotides of length X, where, e.g., X=9, 10, 17, 20, 22, 23, 25, 27, 30 or 35 nucleotides.

The oligonucleotides or oligomers according to the present invention constitute effective tools useful to ascertain genetic and epigenetic parameters of the genomic sequence corresponding to SEQ ID NO:1 to SEQ ID NO:71. Preferred sets of such oligonucleotides or modified oligonucleotides of length X are those consecutively overlapping sets of oligomers corresponding to SEQ ID NO:1 to SEQ ID NO:355 (and to the complements thereof). Preferably, said oligomers comprise at least one CpG, TpG or CpA dinucleotide.

Particularly preferred oligonucleotides or oligomers according to the present invention are those in which the cytosine of the CpG dinucleotide (or of the corresponding converted TpG or CpA dinculeotide) sequences is within the middle third of the oligonucleotide; that is, where the oligonucleotide is, for example, 13 bases in length, the CpG, TpG or CpA dinucleotide is positioned within the fifth to ninth nucleotide from the 5′-end.

The oligonucleotides of the invention can also be modified by chemically linking the oligonucleotide to one or more moieties or conjugates to enhance the activity, stability or detection of the oligonucleotide. Such moieties or conjugates include chromophores, fluorophors, lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. The probes may also exist in the form of a PNA (peptide nucleic acid) which has particularly preferred pairing properties. Thus, the oligonucleotide may include other appended groups such as peptides, and may include hybridization-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a chromophore, fluorophor, peptide, hybridization-triggered cross-linking agent, transport agent, hybridization-triggered cleavage agent, etc.

The oligonucleotide may also comprise at least one art-recognized modified sugar and/or base moiety, or may comprise a modified backbone or non-natural internucleoside linkage.

The oligonucleotides or oligomers according to particular embodiments of the present invention are typically used in ‘sets,’ which contain at least one oligomer for analysis of each of the CpG dinucleotides of genomic sequence SEQ ID NO:1 to SEQ ID NO:71 and sequences complementary thereto, or to the corresponding CpG, TpG or CpA dinucleotide within a sequence of the pretreated nucleic acids according to SEQ ID NO:72 to SEQ ID NO:355 and sequences complementary thereto. However, it is anticipated that for economic or other factors it may be preferable to analyze a limited selection of the CpG dinucleotides within said sequences, and the content of the set of oligonucleotides is altered accordingly.

Therefore, in particular embodiments, the present invention provides a set of at least two (2) (oligonucleotides and/or PNA-oligomers) useful for detecting the cytosine methylation state in pretreated genomic DNA (SEQ ID NO:72 to SEQ ID NO:355), or in genomic DNA (SEQ ID NO:1 to SEQ ID NO:71 and sequences complementary thereto). These probes enable diagnosis, classification and/or therapy of genetic and epigenetic parameters of colon cell proliferative disorders. The set of oligomers may also be used for detecting single nucleotide polymorphisms (SNPs) in pretreated genomic DNA (SEQ ID NO:72 to SEQ ID NO:355), or in genomic DNA (SEQ ID NO:1 to SEQ ID NO:71 and sequences complementary thereto).

In preferred embodiments, at least one, and more preferably all members of a set of oligonucleotides is bound to a solid phase.

In further embodiments, the present invention provides a set of at least two (2) oligonucleotides that are used as ‘primer’ oligonucleotides for amplifying DNA sequences of one of SEQ ID NO:1 to SEQ ID NO:355 and sequences complementary thereto, or segments thereof.

It is anticipated that the oligonucleotides may constitute all or part of an “array” or “DNA chip” (i.e., an arrangement of different oligonucleotides and/or PNA-oligomers bound to a solid phase). Such an array of different oligonucleotide- and/or PNA-oligomer sequences can be characterized, for example, in that it is arranged on the solid phase in the form of a rectangular or hexagonal lattice. The solid-phase surface may comprise, or be composed of silicon, glass, polystyrene, aluminum, steel, iron, copper, nickel, silver, gold, or combinations thereof. Nitrocellulose as well as plastics such as nylon, which can exist in the form of pellets or also as resin matrices, may also be used. An overview of the Prior Art in oligomer array manufacturing can be gathered from a special edition of Nature Genetics (Nature Genetics Supplement, Volume 21, January 1999, and from the literature cited therein). Fluorescently labeled probes are often used for the scanning of immobilized DNA arrays. The simple attachment of Cy3 and Cy5 dyes to the 5′-OH of the specific probe are particularly suitable for fluorescence labels. The detection of the fluorescence of the hybridized probes may be carried out, for example, via a confocal microscope. Cy3 and Cy5 dyes, besides many others, are commercially available.

It is also anticipated that the oligonucleotides, or particular sequences thereof, may constitute all or part of an “virtual array” wherein the oligonucleotides, or particular sequences thereof, are used, for example, as 'specifiers' as part of, or in combination with a diverse population of unique labeled probes to analyze a complex mixture of analytes. Such a method, for example is described in US 2003/0013091 (U.S. Ser. No. 09/898,743, published 16 Jan. 2003). In such methods, enough labels are generated so that each nucleic acid in the complex mixture (i.e., each analyte) can be uniquely bound by a unique label and thus detected (each label is directly counted, resulting in a digital read-out of each molecular species in the mixture).

The present invention further provides a method for ascertaining genetic and/or epigenetic parameters of the genomic sequences according to SEQ ID NO:1 to SEQ ID NO:71 within a subject by analyzing cytosine methylation and single nucleotide polymorphisms. Said method comprising contacting a nucleic acid comprising one or more of SEQ ID NO: 1 to SEQ ID NO:71 in a biological sample obtained from said subject with at least one reagent or a series of reagents, wherein said reagent or series of reagents, distinguishes between methylated and non-methylated CpG dinucleotides within the target nucleic acid.

Preferably, said method comprises the following steps: In the first step, a sample of the tissue to be analysed is obtained. The source may be any suitable source, such as cell lines, histological slides, biopsies, tissue embedded in paraffin, bodily fluids, ejaculate, urine, blood and all possible combinations thereof. The DNA is then extracted or otherwise isolated from the sample. Extraction may be by means that are standard to one skilled in the art, including the use of commercially available kits, detergent lysates, sonification and vortexing with glass beads. Once the nucleic acids have been extracted, the genomic double stranded DNA is used in the analysis.

In the second step of the method, the genomic DNA sample is treated in such a manner that cytosine bases which are unmethylated at the 5′-position are converted to uracil, thymine, or another base which is dissimilar to cytosine in terms of hybridization behavior. This will be understood as ‘pretreatment’ or ‘treatment’ herein.

The above-described treatment of genomic DNA is preferably carried out with bisulfite (hydrogen sulfite, disulfite) and subsequent alkaline hydrolysis which results in a conversion of non-methylated cytosine nucleobases to uracil or to another base which is dissimilar to cytosine in terms of base pairing behavior.

In the third step of the method, fragments of the pretreated DNA are amplified, using sets of primer oligonucleotides according to the present invention, and an amplification enzyme. The amplification of several DNA segments can be carried out simultaneously in one and the same reaction vessel. Typically, the amplification is carried out using a polymerase chain reaction (PCR). The set of primer oligonucleotides includes at least two oligonucleotides whose sequences are each reverse complementary, identical, or hybridize under stringent or highly stringent conditions to an at least 18-base-pair long segment of the base sequences of one or more of SEQ ID NO:72 to SEQ ID NO:355 and sequences complementary thereto.

In an alternate embodiment of the method, the methylation status of preselected CpG positions within the nucleic acid sequences comprising one or more of SEQ ID NO:1 to SEQ ID NO:71 may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primers pairs contain at least one primer which hybridizes to a bisulfite treated CpG dinucleotide. Therefore, the sequence of said primers comprises at least one CpG , TpG or CpA dinucleotide. MSP primers specific for non-methylated DNA contain a “T” at the 3′ position of the C position in the CpG. Preferably, therefore, the base sequence of said primers is required to comprise a sequence having a length of at least 9 nucleotides which hybridizes to a pretreated nucleic acid sequence according to one of SEQ ID NO:72 to SEQ ID NO:355 and sequences complementary thereto, wherein the base sequence of said oligomers comprises at least one CpG, TpG or CpA dinucleotide.

The fragments obtained by means of the amplification can carry a directly or indirectly detectable label. Preferred are labels in the form of fluorescence labels, radionuclides, or detachable molecule fragments having a typical mass which can be detected in a mass spectrometer. Where said labels are mass labels, it is preferred that the labeled amplificates have a single positive or negative net charge, allowing for better detectability in the mass spectrometer. The detection may be carried out and visualized by means of, e.g., matrix assisted laser desorption/ionization mass spectrometry (MALDI) or using electron spray mass spectrometry (ESI).

Matrix Assisted Laser Desorption/Ionization Mass Spectrometry (MALDI-TOF) is a very efficient development for the analysis of biomolecules (Karas & Hillenkamp, Anal Chem., 60:2299-301, 1988). An analyte is embedded in a light-absorbing matrix. The matrix is evaporated by a short laser pulse thus transporting the analyte molecule into the vapour phase in an unfragmented manner. The analyte is ionized by collisions with matrix molecules. An applied voltage accelerates the ions into a field-free flight tube. Due to their different masses, the ions are accelerated at different rates. Smaller ions reach the detector sooner than bigger ones. MALDI-TOF spectrometry is well suited to the analysis of peptides and proteins. The analysis of nucleic acids is somewhat more difficult (Gut & Beck, Current Innovations and Future Trends, 1:147-57, 1995). The sensitivity with respect to nucleic acid analysis is approximately 100-times less than for peptides, and decreases disproportionally with increasing fragment size. Moreover, for nucleic acids having a multiply negatively charged backbone, the ionization process via the matrix is considerably less efficient. In MALDI-TOF spectrometry, the selection of the matrix plays an eminently important role. For desorption of peptides, several very efficient matrixes have been found which produce a very fine crystallisation. There are now several responsive matrixes for DNA, however, the difference in sensitivity between peptides and nucleic acids has not been reduced. This difference in sensitivity can be reduced, however, by chemically modifying the DNA in such a manner that it becomes more similar to a peptide. For example, phosphorothioate nucleic acids, in which the usual phosphates of the backbone are substituted with thiophosphates, can be converted into a charge-neutral DNA using simple alkylation chemistry (Gut & Beck, Nucleic Acids Res. 23: 1367-73, 1995). The coupling of a charge tag to this modified DNA results in an increase in MALDI-TOF sensitivity to the same level as that found for peptides. A further advantage of charge tagging is the increased stability of the analysis against impurities, which makes the detection of unmodified substrates considerably more difficult.

In the fourth step of the method, the amplificates obtained during the third step of the method are analysed in order to ascertain the methylation status of the CpG dinucleotides prior to the treatment.

In embodiments where the amplificates were obtained by means of MSP amplification, the presence or absence of an amplificate is in itself indicative of the methylation state of the CpG positions covered by the primer, according to the base sequences of said primer.

Amplificates obtained by means of both standard and methylation specific PCR may be further analyzed by means of hybridization-based methods such as, but not limited to, array technology and probe based technologies as well as by means of techniques such as sequencing and template directed extension.

In one embodiment of the method, the amplificates synthesised in step three are subsequently hybridized to an array or a set of oligonucleotides and/or PNA probes. In this context, the hybridization takes place in the following manner: the set of probes used during the hybridization is preferably composed of at least 2 oligonucleotides or PNA-oligomers; in the process, the amplificates serve as probes which hybridize to oligonucleotides previously bonded to a solid phase; the non-hybridized fragments are subsequently removed; said oligonucleotides contain at least one base sequence having a length of at least 9 nucleotides which is reverse complementary or identical to a segment of the base sequences specified in the present Sequence Listing; and the segment comprises at least one CpG , TpG or CpA dinucleotide.

In a preferred embodiment, said dinucleotide is present in the central third of the oligomer. For example, wherein the oligomer comprises one CpG dinucleotide, said dinucleotide is preferably the fifth to ninth nucleotide from the 5′-end of a 13-mer. One oligonucleotide exists for the analysis of each CpG dinucleotide within the sequence according to SEQ ID NO:1 to SEQ ID NO:71, and the equivalent positions within SEQ ID NO:72 to SEQ ID NO:355. Said oligonucleotides may also be present in the form of peptide nucleic acids. The non-hybridized amplificates are then removed.

In the final step of the method, the hybridized amplificates are detected. In this context, it is preferred that labels attached to the amplificates are identifiable at each position of the solid phase at which an oligonucleotide sequence is located.

In yet a further embodiment of the method, the genomic methylation status of the CpG positions may be ascertained by means of oligonucleotide probes that are hybridised to the bisulfite treated DNA concurrently with the PCR amplification primers (wherein said primers may either be methylation specific or standard).

A particularly preferred embodiment of this method is the use of fluorescence-based Real Time Quantitative PCR (Heid et al., Genome Res. 6:986-994, 1996; also see U.S. Pat. No. 6,331,393) employing a dual-labeled fluorescent oligonucleotide probe (TaqMan™ PCR, using an ABI Prism 7700 Sequence Detection System, Perkin Elmer Applied Biosystems, Foster City, Calif.). The TaqMan™ PCR reaction employs the use of a nonextendible interrogating oligonucleotide, called a TaqMan™ probe, which, in preferred imbodiments, is designed to hybridize to a GpC-rich sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. For analysis of methylation within nucleic acids subsequent to bisulfite treatment, it is required that the probe be methylation specific, as described in U.S. Pat. No. 6,331,393, (hereby incorporated by reference in its entirety) also known as the MethylLight™ assay. Variations on the TaqMan™ detection methodology that are also suitable for use with the described invention include the use of dual-probe technology (Lightcycler™) or fluorescent amplification primers (Sunrise™ technology). Both these techniques may be adapted in a manner suitable for use with bisulfite treated DNA, and moreover for methylation analysis within CpG dinucleotides.

A further suitable method for the use of probe oligonucleotides for the assessment of methylation by analysis of bisulfite treated nucleic acids comprises the use of blocker oligonucleotides. The use of such blocker oligonucleotides has been described by Yu et al., BioTechniques 23:714-720, 1997. Blocking probe oligonucleotides are hybridized to the bisulfite treated nucleic acid concurrently with the PCR primers. PCR amplification of the nucleic acid is terminated at the 5′ position of the blocking probe, such that amplification of a nucleic acid is suppressed where the complementary sequence to the blocking probe is present. The probes may be designed to hybridize to the bisulfite treated nucleic acid in a methylation status specific manner. For example, for detection of methylated nucleic acids within a population of unmethylated nucleic acids, suppression of the amplification of nucleic acids which are unmethylated at the position in question would be carried out by the use of blocking probes comprising a ‘CpG’ at the position in question, as opposed to a ‘CpA.’

For PCR methods using blocker oligonucleotides, efficient disruption of polymerase-mediated amplification requires that blocker oligonucleotides not be elongated by the polymerase. Preferably, this is achieved through the use of blockers that are 3′-deoxyoligonucleotides, or oligonucleotides derivitized at the 3′ position with other than a “free” hydroxyl group. For example, 3′-O-acetyl oligonucleotides are representative of a preferred class of blocker molecule.

Additionally, polymerase-mediated decomposition of the blocker oligonucleotides should be precluded. Preferably, such preclusion comprises either use of a polymerase lacking 5′-3′ exonuclease activity, or use of modified blocker oligonucleotides having, for example, thioate bridges at the 5′-terminii thereof that render the blocker molecule nuclease-resistant. Particular applications may not require such 5′ modifications of the blocker. For example, if the blocker- and primer-binding sites overlap, thereby precluding binding of the primer (e.g., with excess blocker), degradation of the blocker oligonucleotide will be substantially precluded. This is because the polymerase will not extend the primer toward, and through (in the 5′-3′ direction) the blocker—a process that normally results in degradation of the hybridized blocker oligonucleotide.

A particularly preferred blocker/PCR embodiment, for purposes of the present invention and as implemented herein, comprises the use of peptide nucleic acid (PNA) oligomers as blocking oligonucleotides. Such PNA blocker oligomers are ideally suited, because they are neither decomposed nor extended by the polymerase. In a further preferred embodiment of the method, the fifth step of the method comprises the use of template-directed oligonucleotide extension, such as MS-SNuPE as described by Gonzalgo & Jones, Nucleic Acids Res. 25:2529-2531, 1997.

In yet a further embodiment of the method, the fifth step of the method comprises sequencing and subsequent sequence analysis of the amplificate generated in the third step of the method (Sanger F., et al., Proc Natl Acad Sci USA 74:5463-5467, 1977).

Additional embodiments of the invention provide a method for the analysis of the methylation status of genomic DNA according to the invention (SEQ ID NO: 1 to SEQ ID NO:71, and complements thereof) without the need for pretreatment.

In the first step of such additional embodiments, the genomic DNA sample is isolated from tissue or cellular sources. Preferably, such sources include cell lines, histological slides, body fluids, or tissue embedded in paraffin. In the second step, the genomic DNA is extracted. Extraction may be by means that are standard to one skilled in the art, including but not limited to the use of detergent lysates, sonification and vortexing with glass beads. Once the nucleic acids have been extracted, the genomic double-stranded DNA is used in the analysis.

In a preferred embodiment, the DNA may be cleaved prior to the treatment, and this may be by any means standard in the state of the art, in particular with methylation-sensitive restriction endonucleases.

In the third step, the DNA is then digested with one or more methylation sensitive restriction enzymes. The digestion is carried out such that hydrolysis of the DNA at the restriction site is informative of the methylation status of a specific CpG dinucleotide.

In the fourth step, which is optional but a preferred embodiment, the restriction fragments are amplified. This is preferably carried out using a polymerase chain reaction, and said amplificates may carry suitable detectable labels as discussed above, namely fluorophore labels, radionuclides and mass labels.

In the fifth step the amplificates are detected. The detection may be by any means standard in the art, for example, but not limited to, gel electrophoresis analysis, hybridization analysis, incorporation of detectable tags within the PCR products, DNA array analysis, MALDI or ESI analysis.

In the final step the of the method the presence, absence or subclass of colon cell proliferative disorder is deduced based upon the methylation state of at least one CpG dinucleotide sequence of SEQ ID NO:1 to SEQ ID NO:71, or an average, or a value reflecting an average methylation state of a plurality of CpG dinucleotide sequences of SEQ ID NO:1 to SEQ ID NO:71.

Diagnostic and/or Prognostic Assays for Colon Cell Proliferative Disorders

The present invention enables diagnosis and/or prognosis of events which are disadvantageous to patients or individuals in which important genetic and/or epigenetic parameters within one or more of SEQ ID NO:1 to SEQ ID NO:71 may be used as markers. Said parameters obtained by means of the present invention may be compared to another set of genetic and/or epigenetic parameters, the differences serving as the basis for a diagnosis and/or prognosis of events which are disadvantageous to patients or individuals.

Specifically, the present invention provides for diagnostic and/or prognostic cancer assays based on measurement of differential methylation of one or more CpG dinucleotide sequences of SEQ ID NO:1 to SEQ ID NO:71, or of subregions thereof that comprise such a CpG dinucleotide sequence. Typically, such assays involve obtaining a tissue sample from a test tissue, performing an assay to measure the methylation status of at least one CpG dinucleotide sequence of SEQ ID NO:1 to SEQ ID NO:71 derived from the tissue sample, relative to a control sample, or a known standard, and making a diagnosis or prognosis based, at least in part, thereon.

In particular preferred embodiments, inventive oligomers are used to assess the CpG dinucleotide methylation status, such as those based on SEQ ID NO:1 to SEQ ID NO:355, or arrays thereof, as well as in kits based thereon and useful for the diagnosis and/or prognosis of colon cell proliferative disorders.

Kits

Moreover, an additional aspect of the present invention is a kit comprising, for example: a bisulfite-containing reagent; a set of primer oligonucleotides containing at least two oligonucleotides whose sequences in each case correspond, are complementary, or hybridize under stringent or highly stringent conditions to a 18-base long segment of the sequences SEQ ID NO:1 to SEQ ID NO:355; oligonucleotides and/or PNA-oligomers; as well as instructions for carrying out and evaluating the described method. In a further preferred embodiment, said kit may further comprise standard reagents for performing a CpG position-specific methylation analysis, wherein said analysis comprises one or more of the following techniques: MS-SNuPE, MSP, MethyLight™, HeavyMethyl™, COBRA, and nucleic acid sequencing. However, a kit along the lines of the present invention can also contain only part of the aforementioned components.

While the present invention has been described with specificity in accordance with certain of its preferred embodiments, the following example serves only to illustrate the invention and is not intended to limit the invention within the principles and scope of the broadest interpretations and equivalent configurations thereof.

### EXAMPLES

Pooled genomic DNA from healthy colon, adenomas and colon adenocarcinoma tissue was isolated and analyzed using the discovery methods, AP-PCR and MCA (EXAMPLE 1). These technologies distinguish between methylated and unmethylated CpG sites through the use of methylation sensitive enzymes. In general, whole genomic DNA is first digested to increase manageability, and then further digested with a methylation sensitive enzyme. Methylated fragments are preferentially amplified because cleavage at the unmethylated sites prevents amplification of these products. Differentially methylated fragments identified using these techniques are sequenced (EXAMPLE 2) and compared to the human genome using the BLAST utility in the Ensembl database. The sample set was selected based on the initial aim of the diagnostic problem to be solved. The aim of the study was to enable the identification colon adenocarcinoma and adenomatous polyps in patients, particularly those 50 and older and most preferably by analysis of body fluids. Samples used in the EXAMPLE 1 experiments were divided into three age groups where group A=patients over the age of 65 years, group B=patients ages 50 to 65 and group C=patients younger than 50. Patient samples were also divided depending on the extent of disease. Stage 0 includes normal adjacent tissue (NAT) or no disease, Stage I includes adenomas, Stage 2 includes early carcinoma with no nodal involvement or metastasis (NOM0), and Stage 3 includes advanced disease with nodal involvement and/or metastasis (NIM1). DNA was extracted from snap-frozen patient tissue using Qiagen Genomic tip columns. Up to five DNA samples from each age and stage were pooled and compared as shown in TABLE 1. Multiple comparisons were performed for early and late stage adenocarcinoma for the patients over 65 years of age since this is the group with the highest incidence of colorectal cancer. A single comparison of samples from patients younger than 50 was included to look for overlap of these markers with the other age groups.

TABLE 1
Sample pools used in EXAMPLE 1

| Comparison | Pools |
|---|---|
| A1/A0 | 1 |
| A2/A0 | 3 |
| A3/A0 | 2 |
| B1/B0 | 1 |
| B2/B0 | 1 |
| B3/B0 | 1 |
| C1, 2, 3,/C0 | 1 |
| A1, 2, 3/A0 PBLs | 1 |
| B1, 2, 3/B0 PBLs | 1 |
| C1, 2, 3/C0 PBLs | 1 |

TABLE 2
Samples used According to EXAMPLE 1
(NAT = normal adjacent tissue;
PBL = Peripheral Blood Lymphocytes)

| Pool | Tissue | Diagnosis | Age | Stage |
|---|---|---|---|---|
| Nat pool a1 | Colon | NAT | A | 0 |
| Nat pool a1 | Colon | NAT | A | 0 |
| Nat pool a1 | Colon | NAT | A | 0 |
| Nat pool a2 | Colon | NAT | A | 0 |
| Nat pool a2 | Colon | NAT | A | 0 |
| Nat pool a2 | Colon | NAT | A | 0 |
| Nat pool a2 | Colon | NAT | A | 0 |
| Nat pool a2 | Colon | NAT | A | 0 |
| Nat pool a2 | Colon | NAT | A | 0 |
| Nat pool a3 | Colon | NAT | A | 0 |
| Nat pool a3 | Colon | NAT | A | 0 |
| Nat pool a3 | Colon | NAT | A | 0 |
| Nat pool a3 | Colon | NAT | A | 0 |
| Nat pool a3 | Colon | NAT | A | 0 |
| Nat pool a3 | Colon | NAT | A | 0 |
| Pool a1 | Colon | Tubular adenoma | A | 1 |
| Pool a1 | Colon | Large tubulovillous adenoma | A | 1 |
| Pool a1 | Colon | Villous adenoma of ascending | A | 1 |
| | | colon | | |
| Pool a1 | Colon | Benign Tubulovillous adenoma | A | 1 |
| Pool a1 | Colon | Tubulovillous adenoma | A | 1 |
| Pool a2 | Colon | Infiltrating moderately | A | 2 |
| | | differentiated adenocarcinoma | | |
| | | T?N0M0 | | |
| Pool a2 | Colon | Adenocarcinoma well | A | 2 |
| | | differentiated, T3 N0 M0, | | |
| | | Stage II | | |
| Pool a2 | Colon | Mucinous adenocarcinoma | A | 2 |
| | | T?N0M0 | | |
| Pool a2 | Colon | Invasive mod. Differ. Gr. 2/3 | A | 2 |
| | | adenocarcinoma T3N0M0, | | |
| | | Stage II | | |
| Pool a2 | Colon | Adenocarcinoma, moderately | A | 2 |
| | | differentiated; cecum, N0 T2 | | |
| Pool a2 | Colon | Invasive mod. Differ., Grade 2/3 | A | 2 |
| | | adenoca of sigmoid | | |
| | | T2N0M0, Stage II | | |
| Pool a3 | Colon | Mucinous adenocarcinoma | A | 3 |
| | | low % tumor, T4N1MX | | |
| Pool a3 | Colon | Adenocarcinoma, moderately | A | 3 |
| | | differentiated; mucinous, | | |
| | | N1 T3 | | |
| Pool a3 | Colon | Invasive mod differentiated | A | 3 |
| | | adenocarcinoma Grade 2, | | |
| | | T2N1M0 | | |
| Pool a3 | Colon | Adenocarcinoma, moderately | A | 3 |
| | | differentiated, N2 T3 | | |
| Pool a3 | Colon | Adenocarcinoma, moderately | A | 3 |
| | | differentiated, N1 T2 | | |
| Pool a3 | Colon | Adenocarcinoma, well | A | 3 |
| | | differentiated, N1 T3 | | |
| Pbl pool a | PBL | Normal | A | PBL |
| Pbl pool a | PBL | Normal | A | PBL |
| Pbl pool a | PBL | Normal | A | PBL |
| Pbl pool a | PBL | Normal | A | PBL |
| Nat pool b2 | Colon | NAT | B | 0 |
| Nat pool b2 | Colon | NAT | B | 0 |
| Nat pool b2 | Colon | NAT | B | 0 |
| Nat pool b2 | Colon | NAT | B | 0 |
| Nat pool b2 | Colon | NAT | B | 0 |
| Nat pool b1 | Colon | NAT | B | 0 |
| Pool b1 | Colon | Adenoma, tubulovillous, | B | 1 |
| | | benign dysplasia | | |
| Pool b2 | Colon | Well-differentiated | B | 2 |
| | | adenocarcinoma, T2N0M0 | | |
| | | Stage I | | |
| Pool b2 | Colon | Adenocarcinoma, moderately | B | 2 |
| | | differentiated; sigmoid, | | |
| | | N0 M0 T3; stage II | | |
| Pool b2 | Colon | Adenocarcinoma, moderately | B | 2 |
| | | differentiated, N0 M0 T3; | | |
| | | stage II | | |
| Pool b2 | Colon | Adenocarcinoma moderately | B | 2 |
| | | differentiated T3N0M0, | | |
| | | Stage II | | |
| Pool b2 | Colon | Adenocarcinoma, well | B | 2 |
| | | differentiated, N0 M0 T3; | | |
| | | stage II | | |
| Pbl pool b | PBL | Normal | B | PBL |
| Pbl pool b | PBL | Normal | B | PBL |
| Pbl pool b | PBL | Normal | B | PBL |
| Pbl pool b | PBL | Normal | B | PBL |
| Pbl pool b | PBL | Normal | B | PBL |
| Nat pool c2 | Colon | NAT | C | 0 |
| Nat pool c2 | Colon | NAT | C | 0 |
| Nat pool c2 | Colon | NAT | C | 0 |
| Nat pool c2 | Colon | NAT | C | 0 |
| Nat pool c2 | Colon | NAT | C | 0 |
| Nat pool c3 | Colon | NAT | C | 0 |
| Nat pool c3 | Colon | NAT | C | 0 |
| Nat pool c3 | Colon | NAT | C | 0 |
| Pool c2 | Colon | adenocarcinoma well | C | 2 |
| | | differentiated, T3 N0 | | |
| | | M0, Stage II | | |
| Pool c2 | Colon | Well differentiated | C | 2 |
| | | adenocarcinoma T3N0M0 | | |
| | | stage II | | |
| Pool c2 | Colon | adenocarcinoma well | C | 2 |
| | | differentiated, T3 N0 | | |
| | | M0, Stage II | | |
| Pool c2 | Colon | Moderately differentiated | C | 2 |
| | | adenocarcinoma, T3N0M0, | | |
| | | Stage II | | |
| Pool c2 | Colon | Adenocarcinoma moderately | C | 2 |
| | | differentiated T3N0M0, Stage II | | |
| Pool c3 | Colon | Adenocarcinoma, stage III, well | C | 3 |
| | | differentiated, sigmoid, T3N1M0 | | |
| Pool c3 | Colon | Adenocarcinoma, mucinous, | C | 3 |
| | | N1 M0 T3; stage III | | |
| Pool c3 | Colon | Adenocarcinoma, mucinous, | C | 3 |
| | | grade 2, T3N1M0, stage III | | |
| Pbl pool c | PBL | Normal | C | PBL |
| Pbl pool c | PBL | Normal | C | PBL |
| Pbl pool c | PBL | Normal | C | PBL |
| Pbl pool c | PBL | Normal | C | PBL |
| Pbl pool c | PBL | Normal | C | PBL |

### Example 1

### (Restriction Enzyme Analysis)

Identifying one or more primary differentially methylated CpG dinucleotide sequences using a controlled assay suitable for identifying at least one differentially methylated CpG dinucleotide sequences within the entire genome, or a representative fraction thereof.

All processes were performed on both pooled and/or individual samples, and analysis was carried out using two different Discovery methods; namely, methylated CpG amplification (MCA), and arbitrarily-primed PCR (AP-PCR).

AP-PCR.

AP-PCR analysis was performed on sample classes of genomic DNA as follows:

1. DNA isolation; genomic DNA was isolated from sample classes using the commercially available Wizzard™ kit;

2. Restriction enzyme digestion; each DNA sample was digested with 3 different sets of restriction enzymes for 16 hours at 37° C.: RsaI (recognition site: GTAC); RsaI (recognition site: GTAC) plus HpaII (recognition site: CCGG; sensitive to methylation); and RsaI (recognition site: GTAC ) plus MspI (recognition site: CCGG; insensitive to methylation);

3. AP-PCR analysis; each of the restriction digested DNA samples was amplified with the primer sets (SEQ ID NOS:356-379) according to TABLE 1 at a 40° C. annealing temperature, and with [³²P]-dATP.

4. Polyacrylamide Gel Electrophoresis; 1.6 μl of each AP-PCR sample was loaded on a 5% Polyacrylamide sequencing-size gel, and electrophoresed for 4 hours at 130 Watts, prior to transfer of the gel to chromatography paper, covering the transferred gel with saran wrap, and drying in a gel dryer for a period of about 1-hour;

5. Autoradiographic Film Exposure; film was exposed to dried gels for 20 hours at −80° C., and then developed. Glogos was added to the dried gel and exposure was repeated with new film. The first autorad was retained for records, while the second was used for excising bands; and

6. Bands corresponding to differential methylation were visually identified on the gel. Such bands were excised and the DNA therein was isolated and cloned using the Invitrogen TA Cloning Kit.

TABLE 3
Primers used According to the AP-PCR Protocol
Example 1

| | | |
|---|---|---|
| | SEQUENCE | |
| PRIMER | (5′ to 3′) | SEQ ID NO: |

| | | |
|---|---|---|
| GC1 | GGGCCGCGGC | 356 |
| GC2 | CCCCGCGGGG | 357 |
| GC3 | CGCGGGGGCG | 358 |
| GC4 | GCGCGCCGCG | 359 |
| GC5 | GCGGGGCGGC | 360 |
| G1 | GCGCCGACGT | 361 |
| G2 | CGGGACGCGA | 362 |
| G3 | CCGCGATCGC | 363 |
| G4 | TGGCCGCCGA | 364 |
| G5 | TGCGACGCCG | 365 |
| G6 | ATCCCGCCCG | 366 |
| G7 | GCGCATGCGG | 367 |
| G8 | GCGACGTGCG | 368 |
| G9 | GCCGCGNGNG | 369 |
| G10 | GCCCGCGNNG | 370 |
| APBS1 | AGCGGCCGCG | 371 |
| APBS5 | CTCCCACGCG | 372 |
| APBS7 | GAGGTGCGCG | 373 |
| APBS10 | AGGGGACGCG | 374 |
| APBS11 | GAGAGGCGCG | 375 |
| APBS12 | GCCCCCGCGA | 376 |
| APBS13 | CGGGGCGCGA | 377 |
| APBS17 | GGGGACGCGA | 378 |
| APBS18 | ACCCCACCCG | 379 |

TABLE 4
A Selection of the Results of AP-PCR According to EXAMPLE 1

| | | | | | Tissue | Methylation | Tissue | Methylation |
|---|---|---|---|---|---|---|---|---|
| Experiment | Primer 1 | Primer 2 | Primer 3 | band | Type 1 | state 1 | Type 2 | state 2 |
| colon 4.1 | GC1 | G2 | APBS1 | 1 | colon | hypo | colon | hyper |
| | | | | | nat pool | | pool a1 | |
| | | | | | a1 | | | |
| colon 4.1 | GC4 | G5 | APBS1 | 1 | colon | hypo | colon | hyper |
| | | | | | nat pool | | pool a1 | |
| | | | | | a1 | | | |
| colon 4.2 | GC3 | G6 | APBS7 | 1 | colon | hypo | colon | hyper |
| | | | | | nat pool | | pool a1 | |
| | | | | | a1 | | | |
| colon 4.2 | GC3 | G6 | APBS7 | 2 | colon | hypo | colon | hyper |
| | | | | | nat pool | | pool a1 | |
| | | | | | a1 | | | |
| colon 4.2 | GC4 | G5 | APBS7 | 1 | colon | hypo | colon | hyper |
| | | | | | nat pool | | pool a1 | |
| | | | | | a1 | | | |
| colon 4.2 | GC3 | G1 | APBS10 | 1 | colon | hypo | colon | hyper |
| | | | | | nat pool | | pool a1 | |
| | | | | | a1 | | | |
| colon 4.2 | GC3 | G1 | APBS10 | 2 | colon | hypo | colon | hyper |
| | | | | | nat pool | | pool a1 | |
| | | | | | a1 | | | |
| colon 4.2 | GC4 | G2 | APBS10 | 1 | colon | hyper | colon | hypo |
| | | | | | nat pool | | pool a1 | |
| | | | | | a1 | | | |
| colon 4.5 | GC3 | G5 | APBS13 | 1 | colon | hypo | colon | hyper |
| | | | | | nat pool | | pool a1 | |
| | | | | | a1 | | | |
| colon 4.5 | G3 | G4 | APBS17 | 1 | colon | hypo | colon | hyper |
| | | | | | nat pool | | pool a1 | |
| | | | | | a1 | | | |
| colon 4.5 | G5 | G6 | APBS17 | 1 | colon | hypo | colon | hyper |
| | | | | | nat pool | | pool a1 | |
| | | | | | a1 | | | |
| colon 4.6 | G7 | G8 | APBS13 | 1 | colon | hypo | colon | hyper |
| | | | | | nat pool | | pool a1 | |
| | | | | | a1 | | | |
| colon 4.6 | G8 | G10 | APBS13 | 1 | colon | hypo | colon | hyper |
| | | | | | nat pool | | pool a1 | |
| | | | | | a1 | | | |
| colon 4.6 | G5 | G7 | APBS12 | 1 | colon | hypo | colon | hyper |
| | | | | | nat pool | | pool a1 | |
| | | | | | a1 | | | |
| colon 4.7 | G2 | G4 | APBS12 | 1 | colon | hypo | colon | hyper |
| | | | | | nat pool | | pool a1 | |
| | | | | | a1 | | | |
| colon 4.7 | G1 | G3 | APBS11 | 1 | colon | hypo | colon | hyper |
| | | | | | nat pool | | pool a1 | |
| | | | | | a1 | | | |
| colon 4.7 | G1 | G3 | APBS11 | 2 | colon | hypo | colon | hyper |
| | | | | | nat pool | | pool a1 | |
| | | | | | a1 | | | |
| colon 4.8 | G1 | G8 | APBS10 | 1 | colon | hypo | colon | hyper |
| | | | | | nat pool | | pool a1 | |
| | | | | | a1 | | | |
| colon 4.8 | G5 | G9 | APBS7 | 1 | colon | hyper | colon | hypo |
| | | | | | nat pool | | pool a1 | |
| | | | | | a1 | | | |
| colon 4.8 | G2 | G6 | APBS5 | 1 | colon | hypo | colon | hyper |
| | | | | | nat pool | | pool a1 | |
| | | | | | a1 | | | |
| colon 4.8 | G1 | G5 | APBS5 | 1 | colon | hypo | colon | hyper |
| | | | | | nat pool | | pool a1 | |
| | | | | | a1 | | | |
| colon 4.8 | G4 | G10 | APBS5 | 1 | colon | hypo | colon | hyper |
| | | | | | nat pool | | pool a1 | |
| | | | | | a1 | | | |
| colon 4.9 | G1 | G7 | APBS1 | 1 | colon | hypo | colon | hyper |
| | | | | | nat pool | | pool a1 | |
| | | | | | a1 | | | |
| colon 4.9 | APBS10 | APBS13 | APBS17 | 1 | colon | hypo | colon | hyper |
| | | | | | nat pool | | pool a1 | |
| | | | | | a1 | | | |

MCA.

MCA was used to identify hypermethylated sequences in one population of genomic DNA as compared to a second population by selectively eliminating sequences that do not contain the hypermethylated regions. This was accomplished, as described in detail herein above, by digestion of genomic DNA with a methylation-sensitive enzyme that cleaves un-methylated restriction sites to leave blunt ends, followed by cleavage with an isoschizomer that is methylation insensitive and leaves sticky ends. This is followed by ligation of adaptors, amplicon generation and subtractive hybridization of the tester population with the driver population.

In the initial restriction digestion reactions, 5 μg of each genomic DNA pool was digested with SmaI in a 100 μL reaction overnight at 25° C. in NEB buffer 4+BSA, and 100 units of enzyme (10 μL). The pools were then further digested with Xma I (2 μL=100 U), 6 hours at 37° C.

500 ng of the cleaned-up, digested material was ligated to the adapter-primer RXMA24+RXMA12 (Sequence: RXMA24: AGCACTCTCCAGCCTCTCACCGAC (SEQ ID NO: 380); RXMA12: CCGGGTCGGTGA (SEQ ID NO:381). These were hybridized to create the adapter by heating together at 70° C. and slowly cooling to room temperature (RT) in a 30 μL reaction overnight at 16° C., with 400 U (1 μL) of T4 ligase enzyme.

3 μL of the ligation mix for both tester and driver populations was used in each initial PCR to generate the starting amplicons. Two PCR reactions were run for the tester, and 8 for the driver. Reactions were 100 μL, with 1 μL of 100 μM primer RXMA24 (SEQ ID NO:380), 10 μL PCR buffer,1.2 μL 25 mM dNTPs, 68.8 μl water, 1 μL titanium Taq, 2 μL DMSO, and 10 μL 5 M Betaine. PCR comprised an initial step at 95° C. for 1 minute, followed by 25 cycles at 95° C. for 1 minute, followed by 72° C. for 3 minutes, and a final extension at 72° C. for 10 minutes.

The tester amplicons were then digested with XmaI as described above, yielding overhanging ends, and the driver amplicons were digested with SmaI as above, yielding blunt end fragments.

A new set of adapter primers (hybridized as described for the above RXMA primers) JXMA24+JXMA12 (Sequence: JXMA24: ACCGACGTCGACTATCCATGAACC (SEQ ID NO:382); JXMA12: CCGGGGTTCATG (SEQ ID NO:383) was ligated to the Tester only (using the same conditions as described above for the RXMA primers).

Five μg of digested tester and 40 μg of digested driver amplicons were hybridized in a solution containing 4 μL EE (30 mM EPPS, 3 mM EDTA) and 1 μL of 5 M NaCl at 67° C. for 20 hours. A selective PCR reaction was done using primer JXMA24 (SEQ ID NO:382). The PCR amplification steps were as follows: an initial fill-in step at 72° C. for 5 minutes, followed by 95° C. for 1 minute, and 72° C. for 3 minutes, for 10 cycles. Subsequently, 10 μL of Mung Bean nuclease buffer plus 10 μL Mung Bean Nuclease (10 U) was added and incubated at 30° C. for 30 minutes. This reaction was cleaned up and used as a template for 25 more cycles of PCR using JXMA24 primer (SEQ ID NO:382) and the same conditions.

The resulting PCR product (tester) was digested again using XmaI, as described above, and a third adapter, NXMA24 (AGGCAACTGTGCTATCCGAGTGAC; SEQ ID NO:384)+NXMA12 (CCGGGTCACTCG; SEQ ID NO: 385) was ligated. The tester (500 ng) was hybridized a second time to the original digested driver (40 μg) in 4 μL EE (30 mM EPPS, 3 mM EDTA) and 1 μL 5 M NaCl at 67° C. for 20 hours. Selective PCR was performed using NXMA24 primer (SEQ ID NO:) as follows: an initial fill-in step at 72° C. for 5 minutes, followed by 95° C. for 1 minute, and 72° C. for 3 minutes, for 10 cycles. Subsequently, 10 μL of Mung Bean nuclease buffer plus 10 μL Mung Bean Nuclease (10 U) was added and incubated at 30° C. for 30 minutes. This reaction was cleaned up and used as a template for 25 more cycles of PCR using NXMA24 primer and the same conditions.

The resulting PCR product (1.8 μg) was digested with XmaI (in 50 μL total volume, NEB buffer 4+BSA, and 2 μL=100 U XmaI, 6 hours at 37° C.) and ligated into the vector pBC Sk—predigested with XmaI and phosphatased (675 ng). Five (5) μL of a 30 μL ligation was used to transform chemically competent TOP10™ cells according to the manufacturer's instructions. The transformations were plated onto LB/XGal/IPTG/CAM plates. Selected insert colonies were sequenced according to Example 2.

Scoring of Unique Sequence Embodiments Comprising One or More Differentially Methylated CpG Dinucleotides.

The Discovery methods and comparisons of EXAMPLE 1 resulted in the identification of 712 unique marker sequences. A subset of these sequences were eliminated, because of high (>50%) repeat sequence content. The 509 remaining sequences were further selected according to the following scoring criteria and procedure shown in TABLE 4:

TABLE 4
Scoring Criteria, and ‘Points’ Allotted in view of Same

| | Allotted points |
|---|---|
| Scoring Criterion | if criterion met |
| Appearance (i.e., differentially methylated) | +1 |
| using multiple methods | |
| Appearance in multiple pools | +1 |
| Located within (or comprising) a CpG island | +1 |
| Located within the promoter region of a gene | +1 |
| Near or within predicted or known gene | +1 |
| Known to be associated with disease | +1 |
| Class of gene (transcription factor, growth | +1 |
| factor, etc.) | |
| Repetitive element (negative score) | −8 |

Under this scoring scheme, a MeST sequence receives a point (+1) for satisfaction of each of the above criteria, and receives a score of minus eight (−8) for having repetitive sequence content greater than 50%. The highest score possible is 7, the lowest is (−)8. Scores are automatically generated using a proprietary database. The above-mentioned 509 MeST sequences were further analyzed using the above scoring criteria, along with manual review of the sequences, resulting in identification of a preferred set of 266 unique sequences.

Primers were designed for these 266 sequences for the purpose of bisulfite sequencing. Forty-nine (49) of the sequences were not sequenced for various technical reasons, or changes in scoring according to the above criteria, based on additional information (e.g., updates of the Ensembl database).

### Example 2

### (Bisulfite Sequencing)

For bisulfite sequencing amplification primers were designed to cover each individual sequence when possible or part of the 1000 bp flanking regions surrounding the position. Samples used in Example 1 were utilized for amplicon production in this phase of the study. Ten to fifteen samples each of DNA from normal adjacent colon, colon adenocarcinoma, and normal peripheral blood lymphocytes (PBLs) were treated with sodium bisulfite and sequenced. Initially, sequence data was obtained using MegaBace technology and later sequences were derived using an ABI 3700 device. Traces obtained from sequencing were normalized, and percentage methylation values calculated using an ESME™ analysis program (Epigenomics, A G, Berlin).

Results of Bisulfite Sequencing.

The following properties were noted (screened for):

(1) Bisulfite sequencing indicates differential methylation of a CpG site between selected classes of samples (Fisher score);

(2) Co-methylation is observed;

(3) If only one site has fisher score >1, are there additional sites surrounding with fisher score >0.5?; and

(4) Are there trends in the pattern (e.g., blocks of blue (black) vs. yellow (light grey)), but not necessarily high Fisher score.

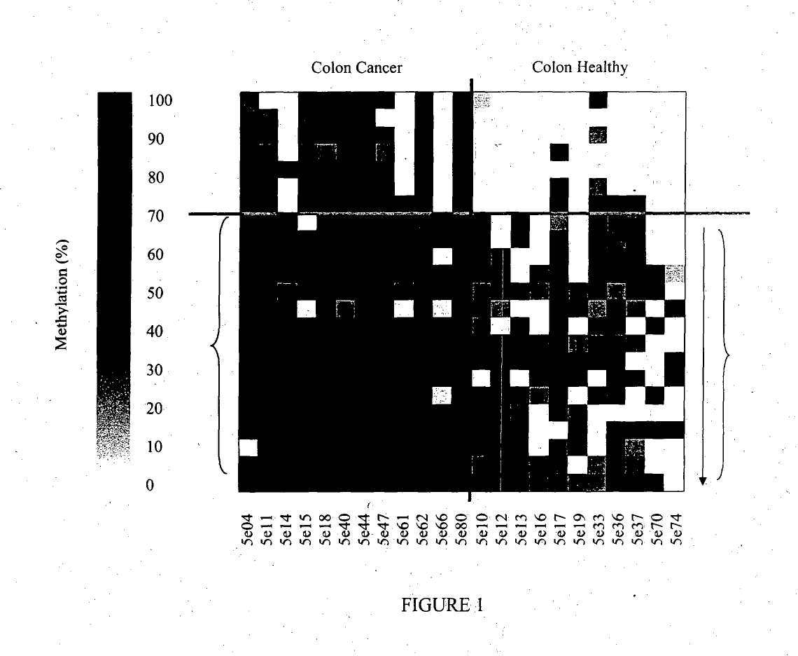

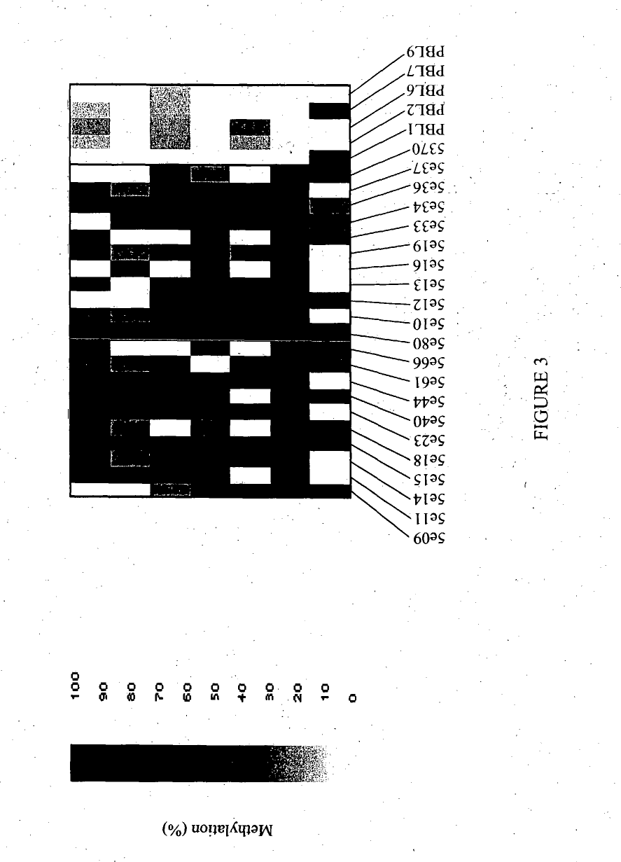

FIGS. 1 though 3 show representative ‘ranked’ matrices produced from bisulfite sequencing data analyzed by means of the proprietary ESME™ program (Epigenetics, A G, Berlin). The overall matrix, in each case, represents the sequencing data for one fragment. Each row of the matrix is a single CpG site within the fragment and each column is an individual DNA sample (sample designations are shown along the X-axis). The bar on the left represents the percent of methylation, with the degree of methylation represented by the darkness of each position within the column from black (Blue) representing 100% methylation to light grey (yellow) representing 0% methylation. Colon cancer samples are shown to the left of the vertical black line, and healthy colon samples are to the right of the vertical black line. In FIG. 3, peripheral blood lymphocytes (PBL) are grouped to the far right of the matrix (i.e., to the right of the second vertical black line).

FIG. 1 represents the sequencing data for a fragment of SEQ ID NO:46 according to EXAMPLE 2 herein below. Each row of the matrix represents a single CpG dinucleotide site within the fragment and each column is an individual DNA sample (sample designations are listed on the X-axis). The vertical calibration bar on the left correlates the intensity of shading or color with the percent of methylation; with the degree of methylation represented by the darkness of each position within the column from black (or blue) representing 100% methylation to light grey (or yellow) representing 0% methylation. Colon cancer samples are to the left of the central vertical black line and healthy colon samples are to the right of the vertical black line. The Figure shows a representative example of a genomic fragment (SEQ ID NO:46 ) exhibiting mosaic patterns of methylation in normal samples, and extensive co-methylation in cancer, positions below the horizontal line (denoted within the limits of the left curly bracket) were considered to be particularly informative.

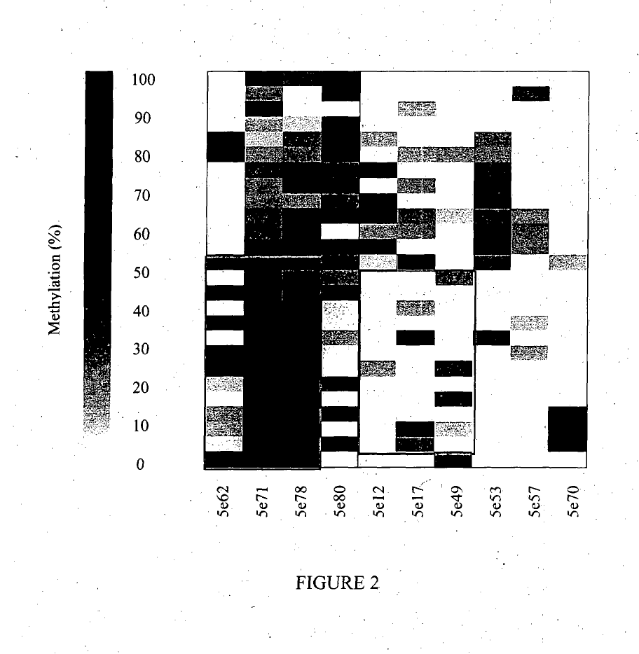

FIG. 2 represents the sequencing data for a fragment of SEQ ID NO:14 according to EXAMPLE 2 herein below. Each row of the matrix represents a single CpG site within the fragment and each column is an individual DNA sample (sample designations are listed on the X-axis). The vertical calibration bar on the left correlates the intensity of shading or color with the percent of methylation; with the degree of methylation represented by the darkness of each position within the column from black (or blue) representing 100% methylation to light grey (or yellow) representing 0% methylation. Colon cancer samples are to the left of the central vertical black line and healthy colon samples are to the right of the central vertical black line. The Figure shows another representative example of a genomic fragment (SEQ ID NO:14) comprising a block of consecutive CpG positions exhibiting differential methylation between cancer (hypermethylated) and normal colon tissue (hypomethylated), denoted by the left and right box frames, respectively.

FIG. 3 represents the sequencing data for a fragment of SEQ ID NO:69 according to EXAMPLE 2 herein below. Each row of the matrix represents a single CpG site within the fragment and each column is an individual DNA sample (sample designations are listed on the X-axis). The vertical calibration bar on the left correlates the intensity of shading or color with the percent of methylation; with the degree of methylation represented by the darkness of each position within the column from black (or blue) representing 100% methylation to light grey (or yellow) representing 0% methylation. Colon cancer samples are to the left of the left vertical black line, healthy colon samples are grouped between the left and right black lines, and peripheral blood lymphocytes (PBL) are grouped to the right of the right black vertical line. The Figure shows a comparison of the methylation patterns between colon tissue (both carcinoma in the left block, and healthy in the central block) and peripheral blood lymphocytes (right block). Colon tissues exhibit hypermethylation in the subject representative fragment (SEQ ID NO:69) as compared to peripheral blood lymphocytes.

We claim:

1. A method for detecting, or detecting and distinguishing between or among colorectal cell proliferative disorders, comprising contacting genomic DNA of a biological sample obtained from the subject with at least one reagent, or series of reagents that distinguishes between methylated and non-methylated CpG dinucleotides within a target sequence of the genomic DNA, wherein the target sequence comprises a sequence of at least contiguous nucleotides of a sequence selected from the group consisting of SEQ ID NOS:1-70, and SEQ ID NO:71.

2. A method according to claim 1, wherein said colorectal cell proliferative disorders are selected from the group consisting of colorectal carcinoma, colon adenomas, and colon polyps.

3. The method according to claim 1, wherein the biological sample obtained from the subject is selected from the group consisting of histological slides, biopsies, paraffin-embedded tissue, bodily fluids, stool, blood, serum, plasma and combinations thereof.

4. A method according to claim 1, comprising:
a) obtaining a biological sample containing genomic DNA;
b) extracting, or otherwise isolating the genomic DNA;
c) digesting the genomic DNA of b) comprising at least one CpG dinucleotide of a sequence selected from the group consisting of SEQ ID NOS:1-70, and SEQ ID NO:71, with one or more methylation sensitive restriction enzymes;
d) detecting the DNA fragments generated in the digest of c);
e) determining, based at least in part on the presence or absence of, or on a property of said fragments, the methylation state of at least one CpG dinucleotide sequence of SEQ ID NO:1 to SEQ ID NO:71, or an average, or a value reflecting an average methylation state of a plurality of CpG dinucleotide sequences of SEQ ID NO:1 to SEQ ID NO:71 , whereby at least one of detecting, or detecting and distinguishing between or among colorectal cell proliferative disorders is, at least in part, enabled.

5. A method according to claim 4, wherein the DNA digest is amplified prior to d).

6. A method according to claim 1, comprising:
a) obtaining, from a subject, a biological sample having subject genomic DNA;
b) treating the genomic DNA, or a fragment thereof, with one or more reagents to convert 5-position unmethylated cytosine bases to uracil or to another base that is detectably dissimilar to cytosine in terms of hybridization properties;
c) contacting the treated genomic DNA, or the treated fragment thereof, with an amplification enzyme and at least two primers comprising, in each case, a contiguous sequence at least 18 nucleotides in length that is complementary to, or hybridizes under moderately stringent or stringent conditions to a sequence selected from the group consisting of SEQ ID NOS:72-355, and complements thereof, wherein the treated DNA or a fragment thereof is either amplified to produce one or more amplificates, or is not amplified; and
d) determining, based on the presence or absence of, or on a property of said amplificate, the methylation state of at least one CpG dinucleotide of a sequence selected from the group consisting of SEQ ID NOS: 1-70, and SEQ ID NO:71, or an average, or a value reflecting an average methylation state of a plurality of said CpG dinucleotide sequences, whereby at least one of detecting, or detecting and distinguishing between or among colorectal cell proliferative disorders is, at least in part, enabled.

7. The method of claim 6, wherein in b) treating the genomic DNA, or the fragment thereof, comprises use of a solution selected from the group consisting of bisulfite, hydrogen sulfite, disulfite, and combinations thereof.

8. The method of claim 6, wherein treating in b) comprises at least one of treatment subsequent to embedding the DNA in agarose, treating in the presence of a DNA denaturing reagent, or treating in the presence of a radical trap reagent.

9. The method of any one of claims 5 or 6, wherein contacting or amplifying comprises use of at least one method selected from the group consisting of: use of a heat-resistant DNA polymerase as the amplification enzyme; use of a polymerase chain reaction (PCR); generation of a amplificate nucleic acid molecule carrying a detectable labels; and combinations thereof.

10. The method of claim 9, wherein the detectable amplificate label is selected from the label group consisting of: fluorescent labels; radionuclides or radiolabels; amplificate mass labels detectable in a mass spectrometer; detachable amplificate fragment mass labels detectable in a mass spectrometer; amplificate, and detachable amplificate fragment mass labels having a single-positive or single-negative net charge detectable in a mass spectrometer; and combinations thereof.

11. A nucleic acid comprising a sequence of at least 18 contiguous nucleotides of a treated genomic DNA sequence selected from the group consisting of SEQ ID NOS:72-355, and sequences complementary thereto, wherein the contiguous sequence comprises at least one CpG, TpA, or CpA dinucleotide, and wherein the treatment is suitable to convert at least one unmethylated cytosine base of the genomic DNA sequence initially to uracil or another base that is detectably dissimilar to cytosine in terms of hybridization.

12. An oligomer or peptide nucleic acid (PNA)-oligomer, said oligomer comprising in each case a sequence of at least 9 contiguous nucleotides that is complementary to, or hybridizes under moderately stringent or stringent conditions to a treated genomic DNA sequence selected from the group consisting of SEQ ID NOS:72-355, and sequences complementary thereto.

13. The oligomer of claim 12, wherein the contiguous sequence includes at least one CpG, TpG or CpA dinucleotide.

14. The oligomer of claim 13, wherein the cytosine of the CpG, the thymine of the TpG, or the adenosine of the CpA dinucleotide is located at about the middle third of the oligomer.

15. A set of oligomers, comprising at least two oligomers according, in each case, to any one of claims 12 to 14.

16. The set of oligomers of claim 15, comprising one or more oligomers suitable for use as primer oligonucleotides for the amplification of a DNA sequence selected from the group consisting of SEQ ID NOS:72-355, and sequences complementary thereto.

17. The set of oligomers of claim 15, wherein at least one oligomer is bound to a solid phase.

18. Use of the set of oligomers according to any one of claims 15 through 17, wherein at least one oligomer can be used as a probe for detecting at least one of the cytosine methylation state, or single nucleotide polymorphisms (SNPs) within a sequence selected from the group consisting of SEQ ID NOS:1-71, and sequences complementary thereto.

19. An oligomer array, according to claim 17.

20. The array of claim 19, wherein the oligomers or peptide nucleic acid (PNA)-oligomers are arranged on a planar solid phase in the form of a rectangular or hexagonal lattice, or in a form substantially so.

21. The array of any one of claims 19 or 20, wherein the solid phase comprises a material selected from the group consisting of silicon, glass, polystyrene, aluminium, steel, iron, copper, nickel, silver, gold, and combinations thereof.

22. A kit for detecting, or for detecting and distinguishing between or among colorectal cell proliferative disorders, comprising:
i) at lease one of a bisulfite reagent or a methylation-sensitive restriction enzyme;
ii) at least one nucleic acid molecule or peptide nucleic acid molecule comprising, in each case, a contiguous sequence of at least 9 nucleotides that is complementary to, or hybridizes under moderately stringent or stringent conditions to a sequence selected from the group consisting of SEQ ID NOS:1-355, and complements thereof.

23. Use of a nucleic acid according to any one of claims 11 or 25, of an oligomer or PNA-oligomer according to any one of claims 12 through 14, of a kit according to claim 22, of an array according to any one of claims 19 through 21, of a set of oligonucleotides according to any one of claims 15 through 17, or of a method according to any one of claims 1 through 10, for classifying, distinguishing between or among, diagnosing or determining the predisposition for colorectal cell proliferative disorders.

24. Use of a nucleic acid according to any one of claims 11 or 25, of an oligomer or PNA-oligomer according to any one of the claims 15 through 17, of a kit according to claim 22, of an array according to any one of the claims 19 through 21, of a set of oligonucleotides according to one of claims 15 through 17, or of a method according to any one of claims 1 through 10, for the therapy of colorectal cell proliferative disorders.

25. An isolated treated nucleic acid derived from a genomic DNA sequence selected from the group consisting of SEQ ID NOS:1-71, and sequences complementary thereto, wherein the treatment is suitable to convert at least one unmethylated cytosine base of the genomic DNA sequence to uracil or to another base that is detectably dissimilar to cytosine in terms of hybridization.
